(19) United States
(12) Patent Application Publication
(10) Pub. No.: US 2024/0016905 A1
(43) Pub. Date: Jan. 18, 2024

(54) METHODS OF TREATING HYPERGLYCEMIA AND SUPPRESSING ONSET OF TYPE 1 DIABETES

(71) Applicants: Aditxt, Inc., Mountain View, CA (US); THE BOARD OF TRUSTEES OF THE LELAND STANFORD JUNIOR UNIVERSITY, Stanford, CA (US)

(72) Inventors: Shahrokh SHABAHANG, Mountain View, CA (US); David ALLEVA, Mountain View, CA (US); Avnesh S. THAKOR, Palo Alto, CA (US)

(21) Appl. No.: 17/909,705

(22) PCT Filed: Mar. 3, 2021
(86) PCT No.: PCT/US2021/020711

§ 371 Date: Sep. 6, 2022

Related U.S. Application Data
(60) Provisional application No. 62/984,661, filed on Mar. 3, 2020.

(51) Int. Cl. A61K 39/00; A61P 3/10

(52) U.S. Cl. CPC A61K 39/0008; A61P 3/10; A61K 2039/577; A61K 2039/53; A61K 2039/55561

(57) ABSTRACT

Methods of reversing hyperglycemia and suppressing diabetes onset in a patient at risk of developing type 1 diabetes are provided. In particular, a vector system comprising a first expression cassette encoding BCL2 associated X apoptosis regulator (BAX) and a hypermethylated second expression cassette encoding a secreted glutamic acid decarboxylase 65 (sGAD55) are administered to the patient to induce a tolerogenic response.

### BACKGROUND

Type 1 diabetes mellitus (T1D) is an autoimmune disease in which insulin-producing I3-cells within pancreatic islets are destroyed by an autoimmune attack coordinated by autoantigen-specific polyclonal T lymphocytes that have escaped control of immune tolerance [1, 2]. The field of immunotherapeutics is addressing defective tolerance processes with immunotherapies that have vaccine-like qualities that avoid unwanted effects characteristic of broad-acting immunosuppressive therapeutics. A promising class of immunotherapies utilize the natural cell death process, apoptosis [3-6], which is a natural non-inflammatory tolerance-inducing pathway. Antigen-presenting cells (APCs), such as dendritic cells (DCs), become tolerogenic after engulfing apoptotic cells; this enables the presentation of processed apoptotic cell autoantigens (without co-stimulation) to regulatory T cells (Tregs) for stimulation or to autoreactive memory effector T cells (Teff) for inactivation [3-6].

There remains a need for the development of efficacious immunotherapies for treatment of autoimmune diseases such as T1D.

### SUMMARY

Methods of reversing hyperglycemia and suppressing diabetes onset in a patient at risk of developing type 1 diabetes are provided. In particular, a vector system comprising (a) a first expression cassette encoding BCL2 associated X apoptosis regulator (BAX); and (b) a hypermethylated second expression cassette encoding a secreted form of glutamic acid decarboxylase 65 (e.g., sGAD55) are administered to the patient, thereby inducing a tolerogenic response, which results in an increase in tolerogenic dendritic cell populations in draining lymph nodes as well as an increase in numbers of GAD-specific regulatory T cells. The methods described herein are efficacious in reversing hyperglycemia and suppressing onset of type 1 diabetes.

In one aspect, a method of reversing hyperglycemia in a patient at risk of developing type 1 diabetes is provided, the method comprising administering a therapeutically effective amount of a vector system comprising (a) a first expression cassette comprising a polynucleotide encoding BAX; and (b) a hypermethylated second expression cassette comprising a polynucleotide encoding a secreted form of glutamic acid decarboxylase 65 (GAD65).

In another aspect, a method of suppressing diabetes onset in a patient at risk of developing type 1 diabetes is provided, the method comprising administering a therapeutically effective amount of a vector system comprising (a) a first expression cassette comprising a polynucleotide encoding BAX; and (b) a hypermethylated second expression cassette comprising a polynucleotide encoding a secreted form of glutamic acid decarboxylase 65 (GAD65).

In yet another aspect, a method of increasing numbers of tolerogenic dendritic cells and GAD-specific regulatory T cells in a patient at risk of developing type 1 diabetes is provided, the method comprising administering an effective amount of a vector system comprising a first expression cassette comprising a polynucleotide encoding BCL2 associated X apoptosis regulator (BAX) and a second expression cassette comprising a hypermethylated polynucleotide encoding a secreted form of glutamic acid decarboxylase 65 (e.g., sGAD55). In any of the aforementioned embodiments, the first expression cassette may further comprise a promoter operably linked to the polynucleotide encoding the BAX and the second expression cassette may further comprise a promoter operably linked to the polynucleotide encoding the secreted form of GAD65. In certain embodiments, the first expression cassette comprises a CMV promoter or an SV-40 promoter operably linked to the polynucleotide encoding the BAX. In certain embodiments, the second expression cassette comprises an SV-40 promoter operably linked to the polynucleotide encoding the secreted form of GAD65.

In any of the aforementioned embodiments, the secreted form of GAD65 may be encoded by msGAD55.

In any of the aforementioned embodiments, the vector system may comprise (a) a first vector comprising the first expression cassette expressing BAX; and (b) a hypermethylated second vector comprising the second expression cassette expressing the secreted form of GAD65. In some embodiments, the first vector and the second vector are administered at a ratio ranging from 1:1 to 1:8, including any ratio within this range such as 1:1, 1:2, 1:3, 1:4, 1:5, 1:6, 1:7, or 1:8. In some embodiments, the first vector and the second vector are administered at a ratio of 1:2.

In any of the aforementioned embodiments, the patient may have mild hyperglycemia, moderate hyperglycemia, or severe hyperglycemia. In certain embodiments, the patient has severe hyperglycemia and the first vector and the second vector are administered at a ratio of 1:2.

In any of the aforementioned embodiments, the patient may have an amount of insulin-producing pancreatic beta cells less than 50%, less than 60%, less than 70%, or less than 80% of a reference amount of beta cells for a non-diabetic subject. In some embodiments, the patient has lost 50% to 80% of the beta cells, including any amount within this range such as 50%, 55%, 60%, 65%, 70%, 75%, or 80% of the beta cells.

In any of the aforementioned embodiments, the patient may be human.

In another aspect, a method of increasing numbers of tolerogenic dendritic cells and GAD-specific regulatory T cells in a patient at risk of developing type 1 diabetes is provided, the method comprising administering an effective amount of a vector system comprising a first expression cassette comprising a polynucleotide encoding BCL2 associated X apoptosis regulator (BAX) and a second expression cassette comprising a hypermethylated polynucleotide encoding a secreted form of glutamic acid decarboxylase 65 (e.g., sGAD55).

### BRIEF DESCRIPTION OF THE DRAWINGS

FIGS. 1A-1D. ADi-100-induced tol-DC subsets in draining lymph nodes of NOD mice. Groups of 8-week-old female NOD mice were vaccinated with vector plasmid DNA alone (control) or ADi-100 1:4 (BAX 10 μg+msGAD 40 μg). After 4 days of vaccination, leukocytes were isolated from draining lymph nodes (inguinal) and DC populations were analyzed via flow cytometry. DC populations were defined phenotypically. FIG. 1A shows total classical DC population, MHC Class II⁺/CD11c⁺. FIG. 1B shows tol-DC lymphoid tissue-resident populations, MHC Class II⁺/CD11c⁺/CD8α⁺ (“CD8α⁺”), MHC Class II⁺/CD11c⁺/CD11b⁺/CD103⁺ (“CD11b⁺/CD103⁺”), and tissue-migratory/Non-lymphoid tissue tol-DC populations, MHC Class II⁺/CD11c⁺/CD207⁺ (“CD207⁺”). FIG. 1C shows plasmacytoid DC population, MHC Class II (IAg⁷)⁺/CD11c⁻/PDCA⁺. *p<0.001 compared to Vector control cohort (2-tailed t test). CD11c⁺ (cDC; CD11c⁺ CD8⁺ Integrin αvβ8⁺) and CD11c⁻ (plasmacytoid DCs, pDC; CD11c−/PDCA⁺) tolerogenic DC populations prepared from splenocytes of vector control- or ADi-100 1:2-treated NOD mice were cultured with GAD-stimulated (3-day) CD4+ T lymphocytes from untreated NOD mice and rhIL-2 for 72 hrs and proliferation was assessed via CSFE staining and flow cytometry (FIG. 1D). Cell division was analyzed using FlowJo software and proliferation was calculated as the percentage of dividing cells per total CD4⁺ T cells.

FIG. 2. Two ADi-100 formulations containing different BAX and msGAD55 content suppressed the incidence of diabetes in NOD mice when treating mild hyperglycemia (≥140 mg/dL). Groups of 8-week-old female NOD mice were monitored weekly for fasting blood glucose (FBG) levels, and on the first day that FBG was ≥140 mg/dL (day 0; mild hyperglycemia), mice received an i.d. injection (50 μL) once per week for 8 weeks of formulations containing 50 μg total of different amounts of empty vectors (V_{b}, BAX vector; mVₐ, hypermethylated antigen vector) and those carrying BAX or msGAD55 [8]. Untreated mice did not receive any injection. The study was terminated once 100% of mice were diagnosed with diabetes in the untreated cohort (i.e., 2 FBG readings 300 mg/dL at least 7 days apart). The percentage of mice that remained free of diabetes in each cohort is presented. Note that raw FBG data per mouse used to calculate disease incidence for the first five cohorts (but not ADi-100 1:2) were obtained from data sets that appeared in our previous publication [8], but which were only presented as raw FBG data in a longitudinal format (mouse age); i.e., here, the data are represented in the form of “diabetes incidence” that includes the additional ADi-100 1:2 data that were not included in the previous publication. * p<0.001 compared to untreated cohort.

FIG. 3. Increased efficacy of ADi-100 containing greater BAX plasmid content when administered to highly hyperglycemic NOD mice. Groups of female NOD mice were monitored weekly for morning blood glucose (mBG) levels, in which each mouse received the first ADi-100 dose (day 0) of an i.d. injection of either of two ADi-100 formulations, 1:4 or 1:2, when mBG was ≥180 mg/dL on at least two occasions or upon the first occurrence of mBG≥200 mg/dL. On day 0, the mean±SEM mBG of all 31 mice was 244±12 mg/dL. Mice received weekly ADi-100 injections thereafter for a total of five injections. Daily mBG monitoring continued and mice were diagnosed with diabetes when ≥300 mg/dL on two occasions at least seven days apart. The percentage of mice that remained free of diabetes in each cohort are presented. * p<0.035 for ADi-100 1:4 and p<0.001 for ADi-100 1:2 compared to untreated cohort.

FIG. 4. Immunohistochemical analysis of insulin (upper panels) and hematoxylin and eosin (H&E; insulitis) (lower panels) staining in pancreatic islet samples from representative untreated NOD mice that were non-diabetic (left panels) or diabetic (right panels). Scale bars=50 μm.

### DETAILED DESCRIPTION

Methods of reversing hyperglycemia and suppressing diabetes onset in a patient at risk of developing type 1 diabetes are provided. In particular, a vector system comprising (a) a first expression cassette encoding BCL2 associated X apoptosis regulator (BAX); and (b) a second hypermethylated expression cassette encoding a secreted glutamic acid decarboxylase 65 (e.g., sGAD55) are administered to the patient to induce a tolerogenic response, which may include increasing tolerogenic dendritic cell populations in draining lymph nodes as well as increasing numbers of GAD-specific regulatory T cells. The methods described herein are efficacious in reversing hyperglycemia and suppressing onset of type 1 diabetes.

Before the present compositions, methods, and kits are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

It must be noted that as used herein and in the appended claims, the singular forms “a”, “an”, and “the” include plural referents unless the context clearly dictates otherwise. Thus, for example, reference to “a vector” includes a plurality of such vectors and reference to “the cell” includes reference to one or more cells, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates that may need to be independently confirmed.

### Definitions

“Tolerogenic” means capable of suppressing or down-modulating an adaptive immunological response.

The term “tolerogenic dendritic cell” refers to a dendritic cell that has the ability to induce immunological tolerance. A tolerogenic dendritic cell has low ability to activate effector T cells but high ability to induce and activate regulatory T cells.

“Recombinant” as used herein to describe a nucleic acid molecule means a polynucleotide of genomic, cDNA, viral, semisynthetic, or synthetic origin that, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells,” “host cells,” “cells”, “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence that “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence.

“Expression cassette” or “expression construct” refers to an assembly that is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single stranded DNA (e.g., a M13 origin of replication), at least one multiple cloning site, and a “mammalian” origin of replication (e.g., a SV40 or adenovirus origin of replication).

“Purified polynucleotide” refers to a polynucleotide of interest or fragment thereof that is essentially free, e.g., contains less than about 50%, preferably less than about 70%, and more preferably less than about at least 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material, and includes uptake of peptide- or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

A “reference level” or “reference value” of a biomarker means a level of the biomarker (e.g., blood glucose level or number of pancreatic beta islets) that is indicative of a particular disease state, phenotype, or predisposition to developing a particular disease state or phenotype, or lack thereof, as well as combinations of disease states, phenotypes, or predisposition to developing a particular disease state or phenotype, or lack thereof. A “positive” reference level of a biomarker means a level that is indicative of a particular disease state or phenotype. A “negative” reference level of a biomarker means a level that is indicative of a lack of a particular disease state or phenotype. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a particular disease state, phenotype, or lack thereof in a certain age or gender group). Such reference levels may also be tailored to specific techniques that are used to measure levels of biomarkers in samples (e.g., fluorescence-activated cell sorting (FACS), immunoassays (e.g., ELISA), mass spectrometry (e.g., LC-MS, GC-MS), tandem mass spectrometry, NMR, biochemical or enzymatic assays, PCR, microarray analysis, etc.), where the levels of biomarkers may differ based on the specific technique that is used.

The terms “quantity”, “amount”, and “level” are used interchangeably herein and may refer to an absolute quantification of a molecule, cell (e.g., pancreatic islets), or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression or reversal of the disease and/or symptom(s). Those in need of treatment include those already afflicted (e.g., those with hyperglycemia or pre-diabetic) as well as those in which prevention is desired (e.g., those with increased susceptibility to diabetes, those having a genetic predisposition to developing diabetes, etc.). The terms “treatment”, “treating”, “treat” and the like may encompass suppression of diabetes onset.

The term “suppressing diabetes onset” is a type of treatment used herein to generally refer to preventing or delaying the onset of diabetes. Delaying the onset of diabetes includes delay for one or more days, one or more weeks, one or more months, or longer. Preventing the onset of diabetes includes preventing the onset of diabetes over a specific time period or preventing the onset of diabetes over an indefinite period of time. The onset of diabetes may be identified by any appropriate measurement, such as measurement of blood glucose levels, measurement of insulin production, etc.

Hyperglycemia, as used herein, refers to the condition of having excess glucose in the bloodstream. Hyperglycemia is also referred to as prediabetes or stage 2 disglycemia. Hyperglycemia may be characterized as mild, moderate, or severe, based on blood sugar levels. For people without diabetes, a healthy fasting blood sugar level is about 70 to 100 milligrams per deciliter of blood (mg/dL). Hyperglycemia is diagnosed when fasting blood sugar levels are between about 100 mg/dL and 125 mg/dL. Fasting blood sugar greater than 126 mg/dL indicates the development of clinical diabetes. In the NOD mouse model, mild hyperglycemia refers to hyperglycemia wherein fasting blood glucose levels or morning blood glucose levels are about 140 mg/dL and severe hyperglycemia refers to hyperglycemia wherein fasting blood glucose levels or morning blood glucose levels are about 180 mg/dL or higher. An individual with severe hyperglycemia may also be referred to as “highly hyperglycemic.” Moderate hyperglycemia refers to hyperglycemia wherein fasting or morning blood glucose levels are in the range between mild and severe hyperglycemia, for example, between about 140 mg/dL and about 180 mg/dL in the NOD mouse model.

A therapeutic treatment is one in which the subject is afflicted prior to administration and a prophylactic treatment is one in which the subject is not afflicted prior to administration. In some embodiments, the subject has an increased likelihood of becoming inflicted or is suspected of being afflicted prior to treatment. In some embodiments, the subject is suspected of having an increased likelihood of becoming afflicted. Methods for administration of therapeutic treatments are well known in the art, and include oral, topical, transdermal or intradermal, inhalation, parenteral, sublingual, buccal, rectal, vaginal, and intranasal. The term “parenteral”, as used herein, includes subcutaneous injections (including, for example, transdermal or intradermal injections), intravenous, intramuscular, intrasternal injection or infusion techniques. In certain embodiments, administering comprises administering by a route that is selected from intradermal and mucosal.

The term “about,” particularly in reference to a given quantity, is meant to encompass deviations of plus or minus five percent.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is human.

A “therapeutically effective dose” or “therapeutic dose” is an amount sufficient to effect desired clinical results (i.e., achieve therapeutic efficacy). A therapeutically effective dose can be administered in one or more administrations.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer. Both full-length proteins and fragments thereof are encompassed by the definition. The terms also include postexpression modifications of the polypeptide, for example, phosphorylation, glycosylation, acetylation, hydroxylation, oxidation, and the like.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), and any other type of polynucleotide which is an N- or C-glycoside of a purine or pyrimidine base. There is no intended distinction in length between the terms “polynucleotide,” “oligonucleotide,” “nucleic acid” and “nucleic acid molecule,” and these terms are used interchangeably.

By “isolated” is meant, when referring to a protein, polypeptide, or peptide, that the indicated molecule is separate and discrete from the whole organism with which the molecule is found in nature or is present in the substantial absence of other biological macromolecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The present disclosure provides the following Embodiments.
Embodiment 1. A method of suppressing diabetes onset in a patient at risk of developing type 1 diabetes, the method comprising administering a therapeutically effective amount of a vector system comprising:
   (a) a first expression cassette comprising a polynucleotide encoding BCL2 associated X apoptosis regulator (BAX); and
   (b) a hypermethylated second expression cassette comprising a polynucleotide encoding a secreted form of glutamic acid decarboxylase 65 (GAD65).
Embodiment 2. The method of embodiment 1, wherein the first expression cassette further comprises a promoter operably linked to the polynucleotide encoding the BAX.
Embodiment 3. The method of embodiment 2, wherein the first expression cassette comprises a CMV promoter or an SV-40 promoter operably linked to the polynucleotide encoding the BAX.
Embodiment 4. The method of any one of embodiments 1-3, wherein the second expression cassette further comprises a promoter operably linked to the polynucleotide encoding the secreted form of GAD65.
Embodiment 5. The method of embodiment 4, wherein the second expression cassette comprises an SV-40 promoter operably linked to the polynucleotide encoding the secreted form of GAD65.
Embodiment 6. The method of any one of embodiments 1 to 5, wherein the secreted form of GAD65 is encoded by msGAD55.
Embodiment 7. The method of any one of embodiments 1 to 6, wherein the vector system comprises:
   (a) a first vector comprising the first expression cassette expressing BAX; and
   (b) a hypermethylated second vector comprising the second expression cassette expressing the secreted form of GAD65.
Embodiment 8. The method of embodiment 7, wherein the second vector is hypermethylated at CpG motifs.
Embodiment 9. The method of embodiment 7 or 8, wherein the first vector and the second vector are administered at a ratio ranging from 1:1 to 1:8.
Embodiment 10. The method of embodiment 9, wherein the first vector and the second vector are administered at a ratio of 1:2.
Embodiment 11. The method of any one of embodiments 1 to 10, wherein the patient has mild hyperglycemia, moderate hyperglycemia, or severe hyperglycemia.
Embodiment 12. The method of embodiment 11, wherein the patient has mild hyperglycemia.
Embodiment 13. The method of embodiment 11, wherein the patient has severe hyperglycemia.
Embodiment 14. The method of embodiment 13, wherein the first vector and the second vector are administered at a ratio of 1:2.
Embodiment 15. The method of any one of embodiments 1 to 14, wherein diabetes onset is identified by measurement of blood glucose levels or measurement of insulin production.
Embodiment 16. The method of embodiment 15, wherein the blood glucose levels are fasting blood glucose levels or morning blood glucose levels.
Embodiment 17. The method of any one of embodiments 1 to 16, wherein diabetes onset is delayed for one or more days, for one or more weeks, for one or more months, or longer.
Embodiment 18 The method of any one of embodiments 1 to 17, wherein the patient has an amount of insulin-producing pancreatic beta cells less than 50% of a reference amount of pancreatic beta cells for a non-diabetic subject.
Embodiment 19. The method of embodiment 18, wherein the patient has lost 50% to 80% of the insulin-producing pancreatic beta cells.
Embodiment 20. The method of any one of embodiments 1 to 19, wherein the administration results in an increase in the numbers of tolerogenic dendritic cells and/or GAD-specific regulatory T cells.
Embodiment 21. The method of embodiment 20, wherein the proportion of CD8α⁺ tolerogenic dendritic cells to the total CD11c⁺ dendritic cell population in draining inguinal lymph nodes is increased about 13-fold.
Embodiment 22. The method of embodiment 20, wherein the proportion of CD11b⁺/CD103⁺ tolerogenic dendritic cells to the total CD11c⁺ dendritic cell population in draining inguinal lymph nodes is increased about 2-fold.
Embodiment 23. The method of embodiment 20, wherein the proportion of CD207⁺ tolerogenic dendritic cells to the total CD11c⁺ dendritic cell population in draining inguinal lymph nodes is increased about 2.5-fold.
Embodiment 24. The method of any one of embodiments 1 to 23, wherein the patient is human.
Embodiment 25. The method of any one of embodiments 1 to 24, wherein the vector system is administered intradermally or mucosally.
Embodiment 26. A method of reversing hyperglycemia in a patient at risk of developing type 1 diabetes, the method comprising administering a therapeutically effective amount of a vector system comprising:
   (a) a first expression cassette comprising a polynucleotide encoding BCL2 associated X apoptosis regulator (BAX); and
   (b) a hypermethylated second expression cassette comprising a polynucleotide encoding a secreted form of glutamic acid decarboxylase 65 (GAD65).
Embodiment 27. The method of embodiment 26, wherein the first expression cassette further comprises a promoter operably linked to the polynucleotide encoding the BAX.
Embodiment 28. The method of embodiment 27, wherein the first expression cassette comprises a CMV promoter or an SV-40 promoter operably linked to the polynucleotide encoding the BAX.
Embodiment 29. The method of any one of embodiments 26-28, wherein the second expression cassette further comprises a promoter operably linked to the polynucleotide encoding the secreted form of GAD65.
Embodiment 30. The method of embodiment 29, wherein the second expression cassette comprises an SV-40 promoter operably linked to the polynucleotide encoding the secreted form of GAD65.
Embodiment 31. The method of any one of embodiments 26 to 30, wherein the secreted form of GAD65 is encoded by msGAD55.
Embodiment 32. The method of any one of embodiments 26 to 31, wherein the vector system comprises:
   (a) a first vector comprising the first expression cassette expressing BAX; and
   (b) a hypermethylated second vector comprising the second expression cassette expressing the secreted form of GAD65.
Embodiment 33. The method of embodiment 32, wherein the second vector is hypermethylated at CpG motifs.
Embodiment 34. The method of embodiment 32 or 33, wherein the first vector and the second vector are administered at a ratio ranging from 1:1 to 1:8.
Embodiment 35. The method of embodiment 34, wherein the first vector and the second vector are administered at a ratio of 1:2.
Embodiment 36. The method of any one of embodiments 26 to 35, wherein the patient has mild hyperglycemia, moderate hyperglycemia, or severe hyperglycemia.
Embodiment 37. The method of embodiment 36, wherein the patient has mild hyperglycemia.
Embodiment 38. The method of embodiment 36, wherein the patient has severe hyperglycemia.
Embodiment 39. The method of embodiment 38, wherein the first vector and the second vector are administered at a ratio of 1:2.
Embodiment 40. The method of any one of embodiments 26 to 39, wherein the patient has an amount of insulin-producing pancreatic beta cells less than 50% of a reference amount of pancreatic beta cells for a non-diabetic subject.
Embodiment 41. The method of embodiment 40, wherein the patient has lost 50% to 80% of the insulin-producing pancreatic beta cells.
Embodiment 42. The method of any one of embodiments 26 to 41, wherein administration results in an increase in the numbers of tolerogenic dendritic cells and/or GAD-specific regulatory T cells.
Embodiment 43. The method of embodiment 42, wherein the proportion of CD8α⁺ tolerogenic dendritic cells to the total CD11c⁺ dendritic cell population in draining inguinal lymph nodes is increased about 13-fold.
Embodiment 44. The method of embodiment 42, wherein the proportion of CD11b⁺/CD103⁺ tolerogenic dendritic cells to the total CD11c⁺ dendritic cell population in draining inguinal lymph nodes is increased about 2-fold.
Embodiment 45. The method of embodiment 42, wherein the proportion of CD207⁺ tolerogenic dendritic cells to the total CD11c⁺ dendritic cell population in draining inguinal lymph nodes is increased about 2.5-fold.
Embodiment 46. The method of any one of embodiments 26 to 45, wherein the patient is human.
Embodiment 47. The method of any one of embodiments 26 to 46, wherein the vector system is administered intradermally or mucosally.
Embodiment 48. A method of increasing numbers of tolerogenic dendritic cells and GAD-specific regulatory T cells in a patient at risk of developing type 1 diabetes, the method comprising administering an effective amount of a vector system comprising:
   (a) a first expression cassette expressing BCL2 associated X apoptosis regulator (BAX); and
   (b) a hypermethylated second expression cassette expressing a secreted form of glutamic acid decarboxylase 65.
Embodiment 49. The method of embodiment 48, wherein the first expression cassette further comprises a promoter operably linked to the polynucleotide encoding the BAX.
Embodiment 50. The method of embodiment 49, wherein the first expression cassette comprises a CMV promoter or an SV-40 promoter operably linked to the polynucleotide encoding the BAX.
Embodiment 51. The method of any one of embodiments 48 to 50, wherein the second expression cassette further comprises a promoter operably linked to the polynucleotide encoding the secreted form of GAD65.
Embodiment 52. The method of embodiment 51, wherein the second expression cassette comprises an SV-40 promoter operably linked to the polynucleotide encoding the secreted form of GAD65.
Embodiment 53. The method of any one of embodiments 48 to 52, wherein the secreted form of GAD65 is encoded by msGAD55.
Embodiment 54. The method of any one of embodiments 48 to 53, wherein the vector system comprises:
   (a) a first vector comprising the first expression cassette expressing BAX; and
   (b) a hypermethylated second vector comprising the second expression cassette expressing the secreted form of GAD65.
Embodiment 55. The method of embodiment 54, wherein the second vector is hypermethylated at CpG motifs.
Embodiment 56. The method of embodiment 54 or 55, wherein the first vector and the second vector are administered at a ratio ranging from 1:1 to 1:8.
Embodiment 57. The method of embodiment 56, wherein the first vector and the second vector are administered at a ratio of 1:2.
Embodiment 58. The method of any one of embodiments 48 to 57, wherein the proportion of CD8α⁺ tolerogenic dendritic cells to the total CD11c⁺ dendritic cell population in draining inguinal lymph nodes is increased about 13-fold.
Embodiment 59. The method of any one of embodiments 48 to 58, wherein the proportion of CD11b⁺/CD103⁺ tolerogenic dendritic cells to the total CD11c⁺ dendritic cell population in draining inguinal lymph nodes is increased about 2-fold.
Embodiment 60. The method of any one of embodiments 48 to 59, wherein the proportion of CD207⁺ tolerogenic dendritic cells to the total CD11c⁺ dendritic cell population in draining inguinal lymph nodes is increased about 2.5-fold.
Embodiment 61. The method of any one of embodiment 48 to 60, wherein the patient is human.
Embodiment 62. The method of any one of embodiment 48 to 61, wherein the vector system is administered intradermally or mucosally.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

It will be apparent to one of ordinary skill in the art that various changes and modifications can be made without departing from the spirit or scope of the invention.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

Reversal of Hyperglycemia and Suppression of Type 1 Diabetes in the NOD Mouse with Apoptotic DNA Immunotherapy

A unique and potent immunotherapy, ADi-100, was developed that consists of two DNA plasmids, one expressing the intracellular apoptosis-inducing signaling molecule, BAX, and the other expressing the islet autoantigen, secreted glutamic acid decarboxylase 65 (sGAD55) [3,7,8]. It was previously shown that the efficacy of ADi-100 in the non-obese diabetic (NOD) mouse model of T1D is significantly increased if the sGAD55 plasmid is hyper-methylated [8], which may reduce inflammation caused by unmethylated CpG motifs that are ligands for the Toll-like receptor 9 expressed on some APCs. ADi-100 treatment also increases sGAD-specific Treg levels in draining lymph nodes of NOD mice along with total CD11c⁺ DCs [7-9]; though it is not known whether these DCs have a tolerogenic phenotype. The present inventors have found that ADi-100 treatment increases tolerogenic DCs (tol-DCs), and increasing the apoptosis-inducing BAX content enhances the efficacy in reversing hyperglycemia when administered to NOD mice during late hyperglycemia, a pre-diabetes stage that has relevance to the corresponding clinical diagnosis stage in human T1D.

### ADi-100: Plasmid DNA Construct

The two DNA plasmids that comprise the ADi-100 formulation previously described [8] are pND2-BAX containing a bax cDNA sequence under transcriptional control of the CMV promoter and pSG5-GAD55 containing a cDNA construct encoding a secreted form of human GAD65 (sGAD55) under transcriptional control of the SV-40 promoter in the pSG5 vector (Stratagene, San Diego, CA, USA). The pSG5-GAD plasmid was hyper-methylated at CpG motifs (msGAD55) in Escherichia coli strain, ER1821, via the activity of SssI methylase (New England BioLabs, Ipswich, MA, USA). This method has been shown to result in 85%-100% methylation of CpG motifs in a plasmid (see Jimenez-Useche et al., Biophys J. 107(7) 1629-1636). Plasmid DNA was dissolved in sterile saline immediately prior to intradermal (i.d.) injection. All plasmids containing the bax sequence insert showed significant and substantial degrees of apoptosis of human HeLa cells (using 1 ug/mL DNA in cultures; data not shown), confirming the activity of the BAX-induced apoptosis tolerance delivery system of ADi-100.

### Animals

Eight-week-old female NOD mice were purchased from Taconic Farms (NOD/MrkTac; Germantown, NY, USA) for studies at Loma Linda University (Loma Linda, CA, USA) [8] and from The Jackson Laboratory (NOD/ShiLtJ; Sacramento, CA, USA) for studies at Stanford University (Palo Alto, CA, USA). All animals were housed in vivariums under pathogen-free conditions at their respective locations and experimentation was approved by the respective Institutional Animal Care and Use Committees.

### Dendritic Cell Isolation and Characterization

Eight-week-old female NOD mice (Taconic Farms) received 2 i.d. injections of 50 μg plasmid DNA alone (vector) or ADi-100 1:4 (BAX 10 μg+msGAD 40 μg) in the abdominal flank region 7 days apart, and leukocytes were isolated from draining inguinal lymph nodes 4 days after the second injection, at which time single-cell suspensions were prepared for analysis of various DC phenotypic populations via flow cytometry. These freshly isolated cells (10 6) were incubated with one or more of the following conjugated antibodies (1 μg; see below) for 30 min on ice and evaluated using a FACSCalibur (BD Biosciences, Franklin Lakes, NJ, USA) as previously described [7]; rat anti-mouse CD317/PDCA-1, clone 129C1, PE-conjugated (BioLegend, San Diego, CA, USA); hamster anti-mouse CD11c, clone N418, FITC-conjugated (BioLegend, San Diego, CA, USA); rat anti-mouse MHC Class II, clone M5/114.15.2, APC-conjugated (R&D Systems, Minneapolis, MN, USA); rat anti-mouse CD8a, clone 53-6.7, PE-conjugated (BioLegend, San Diego, CA, USA); rat anti-mouse Integrin αM/CD11b, Clone M1/70, Alexa Fluor 647 conjugated (R&D Systems, Minneapolis, MN, USA); rat anti-mouse CD103, Clone M290, PE-conjugated (BD Biosciences, Franklin Lakes, NJ, USA); rat anti-mouse CD207, Clone 4C7, PE-conjugated (BioLegend, San Diego, CA, USA).

### GAD-Specific T Lymphocyte Proliferation

To evaluate the ADi-100-induced tolerogenic properties of DCs, pooled splenocytes from eight ADi-100-vaccinated NOD mice (as described above) were used to isolate CD11c⁺ (cDC; CD11c⁺ CD8⁺ Integrin αvβ8⁺) and CD11c⁻ (plasmacytoid DCs, pDC; CD11c−/PDCA+) tol-DC populations using the CD11c positive and mPDCA-1 positive kits (Miltenyi, Auburn, CA, USA), respectively. GAD-stimulated CD4+ lymphocytes were generated by culturing 10⁶ lymph node cells from 8-week-old female NOD mice with GAD (20 μg/mL) in 1 mL of culture medium (Dulbecco's modified Eagle's medium with high glucose, DMEM; Sigma, St. Louis, MO, USA) supplemented with 10% heat-inactivated fetal bovine serum (FBS; HyClone, Logan, UT, USA), 2 mM L-glutamine, 1 mM sodium pyruvate, 0.11 mM sodium bicarbonate] for 3 days, after which CD4+ T cells were enriched as untouched cells using negative selection with anti-CD8, -CD11b, -CD16, -CD56, -CD19, and -CD36 mAbs (Miltenyi Biotec, Auburn, CA, USA), as previously described [7]. T cell purity assessed via flow cytometry was >95% (data not shown). GAD-stimulated CD4+ T cells were stained with 1.5 uM CFSE (Invitrogen, Carlsbad, CA, USA) prior to culture with DCs. DCs (5×10⁴) were cultured with CD4⁺ T cells (5×10⁴) and hrIL-2 (20 U/mL; PeproTech, Rocky Hill, NJ, USA) in the presence or absence of sGAD (20 μg/mL,) in triplicate wells of 96-well plates. After 72 h of culture, anti-CD4-PE mAb and the green nucleic acid stain dead cell-indicator, SYTOX®, (Invitrogen, Carlsbad, CA, USA) were used to detect CFSE⁺CD4⁺SYTOX⁻ cell proliferation via flow cytometry per the manufacturer's instructions. FlowJo 7.6.5 software (Becton, Dickinson, & Co., Ashland, OR, USA) was used to analyze proliferation data, and the percentage of divided CD4+ T cells represents the degree of proliferation. The percentage of divided cells in the absence of sGAD antigen was <1% (not shown).

### Diabetes Studies in NOD Mice

Two NOD mouse diabetes studies were performed by two separate laboratories, respectively, to demonstrate the robustness of ADi-100 efficacy: The first study with mildly hyperglycemic female NOD mice was performed at Loma Linda University (Loma Linda, CA, USA) [8] and the second study with highly hyperglycemic female NOD mice was performed at Stanford University (Palo Alto, CA, USA). All animals were purchased at 8 weeks of age and blood glucose levels were monitored weekly with a glucometer (Bayer Contour Glucose Meter; Ascensia Diabetes Care, Parsippany, NJ, USA) as previously described [8]. Upon the first reading ≥140 mg/dL (fasting blood glucose, FBG, mildly hyperglycemic study) or upon at least two readings ≥180 mg/dL or upon the first occurrence ≥200 mg/dL (morning blood glucose, mBG, highly hyperglycemic study), the animals were randomly assigned to cohorts to receive the first weekly injection of ADi-100 (50 μg) or control vectors. Animals received 50 μL i.d. injections into the abdominal flank as previously described [8], and blood glucose levels were monitored weekly in which diabetes was diagnosed when blood glucose was ≥300 mg/dL on two occasions at least 7 days apart. In the mildly hyperglycemic study, each diabetic mouse was euthanized when FBG reached ≥600 mg/dL, and those that were diabetes-free were euthanized at 50 weeks of age. In the highly hyperglycemic study, all animals were euthanized at 5 weeks post treatment to obtain and compare tissue samples at the same time point. Because the mildly and highly hyperglycemic studies entailed blood glucose assessments as FBG and mBG, respectively, the true mean and SEM difference was calculated. This was determined to be 17.9±10 mg/dL, with FBG being intuitively less than the respective mBG reading due to fasting (two mBG readings were assessed the day before and the day after the respective FBG reading, which was evaluated once per week for 7 weeks for each of the 4 non-diabetic mice; i.e., a total of 28 FBG readings and 56 mBG readings that yielded 56 Δ values was used in deriving the mean difference).

### Immunohistochemistry

Animals were euthanized at the end of the experiment and pancreata harvested, embedded in OCT compound (Tissue Tek, Torrance, CA, USA) or paraffin, and stained for insulin using a rat anti-insulin primary antibody ((1:100, #MAB1417, R & D systems, Minneapolis, MN, USA) and a donkey anti-rat IgG secondary antibody conjugated to Alexa488 (1:500, Invitrogen, Carlsbad, CA, USA) as previously described [10].

### Statistical Analysis

Kaplan-Meier estimates of the disease-free survival curves were plotted and differences among groups were tested by log rank test. Comparisons of continuous variables between groups were performed with Wilcoxon tests; comparisons of categorical variables were performed with Fisher's exact test. All data were analyzed with Stata Release 15.2 (StataCorp LP, College Station, TX, USA). A significance level of 0.05 was used. The two-tailed t test (Prism, GraphPad Software, Inc, San Diego, CA, USA) was used to compare means.

Tol-DC Subset Analysis in Draining Lymph Nodes after ADi-100 Treatment

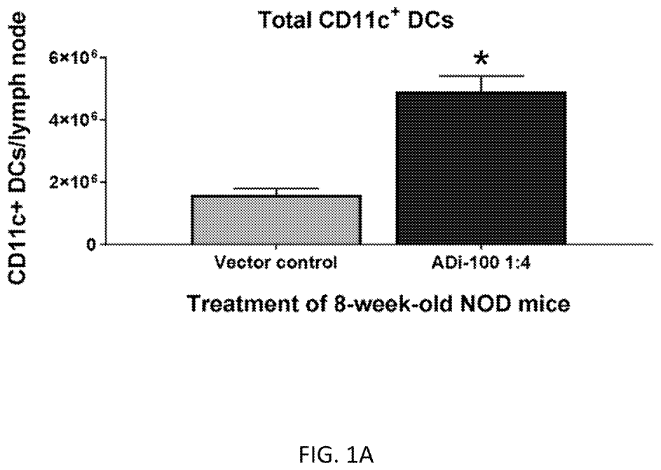

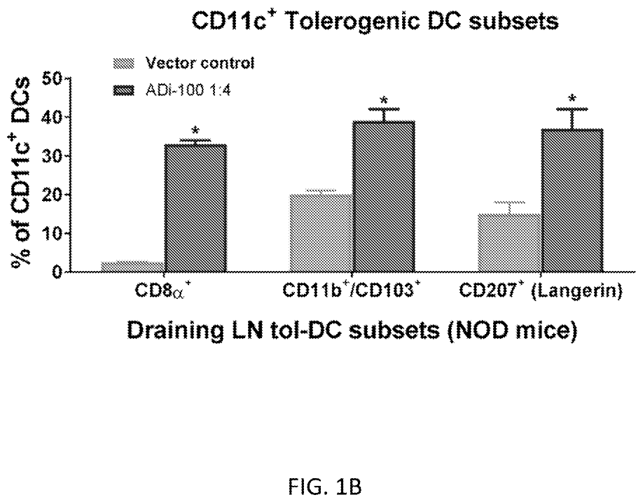

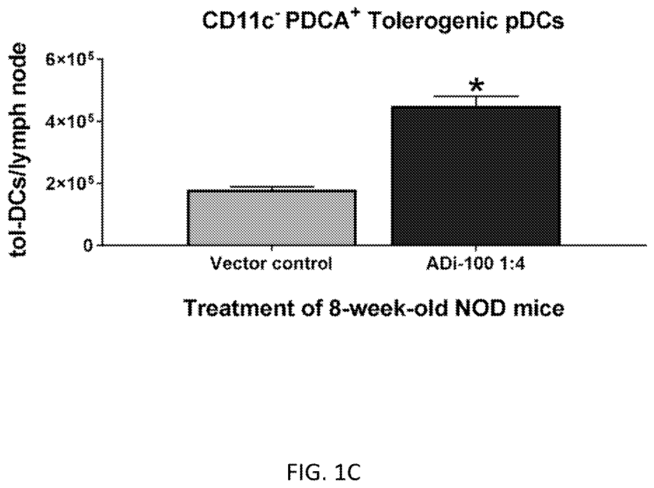

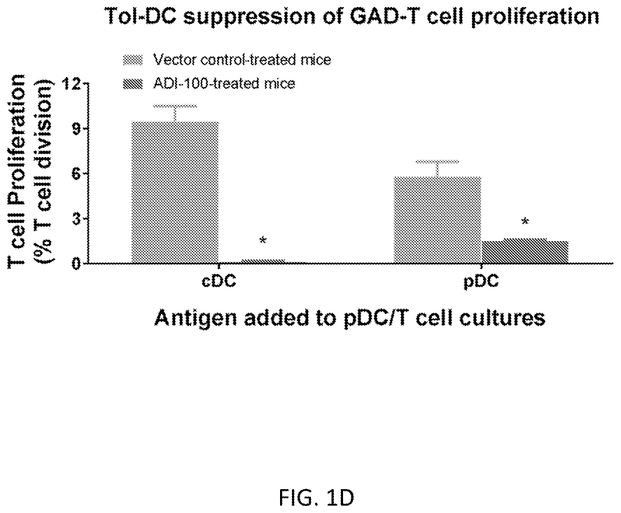

The BAX component of ADi-100 was designed to induce tol-DC migration to draining lymph nodes that subsequently present antigen to stimulate GAD-specific Treg cell numbers and function. Indeed, it has previously been shown that delivery of a plasmid containing BAX and sGAD55 induced functional GAD-specific Treg cells in draining lymph nodes in NOD mice [7], in addition to increasing the number of total CD11c⁺ DCs in draining lymph nodes and spleen [9]. Here, we further defined the “tolerogenic” phenotypes of such DCs (different tol-DC phenotypes reviewed in [11,12]) by evaluating tol-DC populations four days after the second of two weekly injections of ADi-100 1:4 via flow cytometric analysis of draining inguinal lymph nodes (see FIG. 1). While it was confirmed that the total CD11c⁺/MHC class II⁺ DC population per lymph node increased by 3-fold (FIG. 1A), strikingly, the CD8α⁺ tol-DC proportion of the total CD11c⁺ population increased by 13-fold, while the CD11b⁺/CD103⁺ and CD207⁺ tol-DC proportions of the CD11c⁺ population increased by 2- and 2.5-fold, respectively (FIG. 1B). Furthermore, the number of tolerogenic plasmacytoid DCs (pDC; CD11c⁻/PDCA⁺) per lymph node increased by 2.5-fold (FIG. 1C). These results demonstrate that ADi-100 significantly and substantially increased tol-DC migration to the inguinal draining lymph nodes of the abdominal flank injection site. These phenotypically defined DC populations were further evaluated for tolerogenic activity on GAD-specific CD4+ T lymphocyte proliferation. Both CD11c⁺ (cDC; CD11c⁺CD8⁺ Integrin αvβ8⁺) and CD11c⁻ (plasmacytoid DCs, pDC; CD11c−/PDCA⁺) tol-DC populations prepared from splenocytes of vector control- or ADi-100-treated NOD mice lost their ability to support proliferation of GAD-stimulated CD4⁺ T lymphocytes (FIG. 1D), consistent with a tolerogenic phenotype.

### Efficacy of ADi-100 Containing Increased BAX Plasmid Content to Reverse Hyperglycemia in Mildly Hyperglycemic NOD Mice

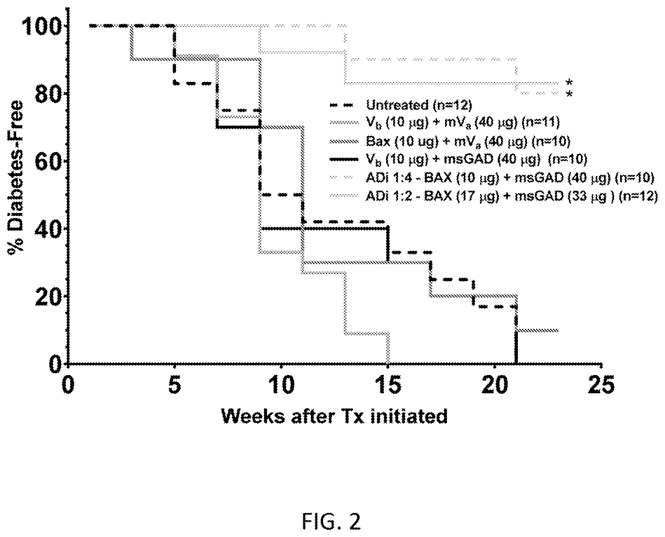

Since BAX-induced apoptosis enhances immune tolerance, we evaluated whether increasing BAX plasmid content of the ADi-100 formulation could enhance the efficacy in reversing hyperglycemia in mildly hyperglycemic female NOD mice. Two ADi-100 formulations containing BAX content at a lower amount (10 μg BAX plasmid and 40 μg msGAD55 plasmid; i.e., 1:4 ratio of BAX:msGAD55) or a higher amount (17 μg BAX+33 msGAD55, 1:2 ratio, 50 μg total) were administered when FBG >140 mg/dL. While the untreated and the empty vector treated cohorts reached 100% diabetes incidence (except the mVa+BAX that reached 90%), there was a significantly lower incidence of diabetes in the ADi-100 1:2- and 1:4-treated cohorts of only 20% at 15 and 23 weeks, respectively (see FIG. 2). None of the ADi-100-treated mice developed diabetes during the first eight weeks during ADi-100 administration. As there were no differences in efficacy between the ADi-100 1:4 and 1:2 formulations administered at a relatively early disease stage of mild hyperglycemia with a mean±SEM FBG of 173±4 mg/dL at day 0 of treatment, possible differences in efficacy were evaluated by treating mice later in the disease process at significantly higher glycemic levels.

### Increased BAX Plasmid Content in the ADi-100 1:2 Formulation Resulted in Greater Efficacy in a Later Stage of Autoimmune Diabetes (i.e., Highly Hyperglycemia)

A challenge in treating NOD mice to reverse hyperglycemia and suppress diabetes onset is to ensure that only mice likely to develop diabetes are treated, and that the timing of treatment is within the “pre-symptomatic” hyperglycemic stage just prior to disease onset when the extent of β-cell loss still permits reversal of hyperglycemia. A hyperglycemic threshold of 4×SD above the mean of normal mBG levels of our aged diabetes-free female mice was derived (mean±SD, 113±17 mg/dL; n=685 daily mBG readings from five naturally diabetes-free mice; note that 20% of our colony remain diabetes-free), which was 180 mg/dL. A non-diabetic mouse is highly unlikely to have a mBG reading above this level (p=0.00003). Indeed, Mathews et al. [13] recently recommended that mBG values should be used instead of FBG to avoid any untoward influence of fasting on the course of disease progression. To determine ADi-100 efficacy when administered relatively late in the disease process during high hyperglycemia, each NOD mouse received the first ADi-100 dose when mBG was 180 mg/dL and weekly doses thereafter for a total of five injections. The mean±SEM mBG on day 0 for all 31 mice was 244±12 mg/dL, which was significantly greater than the FBG mean±SEM of 173±4 mg/dL of the mild hyperglycemic study (p<0.001). Note that the inherent difference between FBG and mBG of 18±10 mg/dL does not account for the large differential of these day 0 mean values.

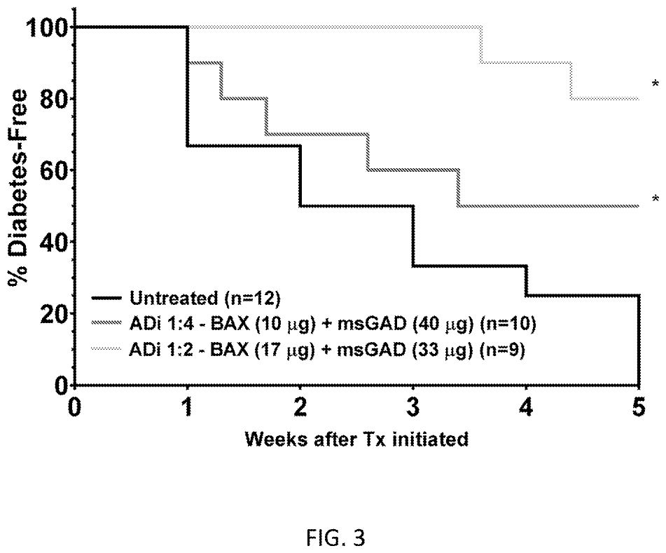

While the untreated cohort progressively developed diabetes, showing an incidence of 100% by five weeks (i.e., day 35, study termination), the ADi-100 1:4 treated cohort showed a 50% suppression of disease incidence from day 17 to the end of the five-week study (see FIG. 3; vs. untreated, p=0.035). Importantly, the ADi-100 1:2 treated cohort showed an 80% suppression of disease incidence from day 31 to the end of the study which was highly significant relative to the untreated group (p=0.001), a statistical significance much greater than that of the ADi-100 1:4 treated group (p=0.035). The probability between the ADi-100 1:2 and 1:4 groups was p=0.17 due to an insufficient number of mice that “converted to diabetes”. It is apparent that the “highly hyperglycemic” acceptance criteria of ≥180 mg/dL led to the initiation of treatment substantially later in the disease process relative to the mildly hyperglycemic study because the time to 100% diabetes incidence in the untreated control groups was substantially faster in the highly hyperglycemic study; 5 vs. 23 weeks, respectively (see FIGS. 2 and 3).

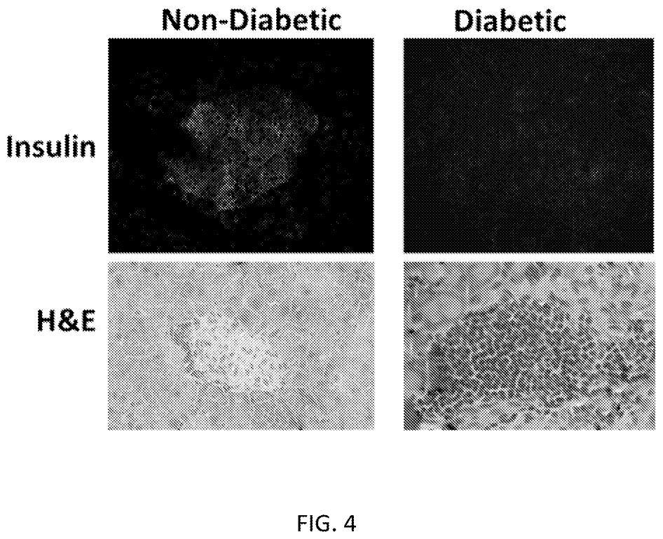

Additional differences exist between the two ADi-100 formulations: (1) Although efficacy in the ADi-100 1:4 group appeared to show a bias of higher mBG day 0 values in the five non-responder mice, this theme did not appear to be the case with the ADi-100 1:2 formulation in which mouse #5 was protected from developing diabetes while having an exceptionally high mBG level of 286 mg/dL on day 0 (see Table 1, below); (2) ADi-100 1:2 appeared to substantially extend the time from day 0 to T1D diagnosis relative to that of ADi-100 1:4 (mean of 4 days for the 1:4 cohort vs. 18 and 29 days for mice #1 and #2 in the 1:2 cohort; see Table 1); (3) mBG levels of all five ADi-100 1:4 diabetic non-responders were ≥600 mg/dL, whereas those of the two from ADi-100 1:2 were controlled below this level at the end of the study (see Table 1); and (4) pancreatic islet insulin expression analysis showed that ADi-100 1:2 responders (i.e., non-diabetic mice at day 35) were positive for insulin, whereas all three of the available samples from ADi-100 1:4 responders were negative (see Table 1; examples of positive and negative insulin staining in FIG. 4). Interestingly, these insulin-negative samples of the three ADi-100 1:4 responders correlated with terminal mBG levels in the hyperglycemic range (≥180 mg/dL), whereas those of the ADi-100 1:2 responders were below this threshold, demonstrating that ADi-100 1:2, but not ADi-100 1:4, reversed hyperglycemia (see Table 1). Thus, this correlation of insulin staining with glycemic levels suggests that responder mice in the ADi-100 1:4 formulation group may ultimately have developed diabetes if followed beyond day 35. Samples from the untreated diabetic control mice and those from all ADi-100 non-responder (diabetic) mice were negative for insulin staining at study termination (see Table 1; some low signals from insulin staining were observed in 2 of 10 untreated diabetic mice at study termination; not shown).

TABLE 1

| | | Age | Occurrences | Occurrences | Age | Days | | mBG at |
|---|---|---|---|---|---|---|---|---|
| | mBG on | (Days) | ≥180 mg/dL | ≥180 mg/dL | (days) | from | Islet | Day |
| Mouse | Tx day 0 | on | Prior to | from Day | at | Day 0 | Insulin | 35 (Study |
| ID | (mg/dL) | Day 0 | Day 0 | 1-35 | T1Dª | to T1D | Staining^{c} | Termination) |

ADi-100 1:4 Tx group
Diabetic “Non-responders”

| | | | | | | | | |
|---|---|---|---|---|---|---|---|---|
| 1 | 245 | 175 | 1 | 35 | 186 | 11 | negative | ≥600 |
| 2 | 230 | 91 | 0 | 35 | 93 | 2 | n/a | ≥600 |
| 3 | 255 | 111 | 0 | 35 | 116 | 5 | negative | ≥600 |
| 4 | 245 | 109 | 0 | 29 | 126 | 2 | negative | ≥600 |
| 5 | 301 | 279 | 3 | 35 | 279 | 0 | negative | ≥600 |
| mean | 255^{b} | 153 | | 34b | 160 | 4 | | |
| SEM | 12 | 35 | | 1 | 34 | 2 | | |

Non-diabetic “Responders”

| | | | | | | | |
|---|---|---|---|---|---|---|---|
| 6 | 217 | 181 | 2 | 15 | n.d. | n/a | |
| 7 | 197 | 270 | 0 | 15 | n.d. | negative | 184 |
| 8 | 192 | 231 | 6 | 16 | n.d. | negative | 199 |
| 9 | 180 | 222 | 1 | 11 | n.d. | negative | 183 |
| 10 | 213 | 146 | 2 | 4 | n.d. | n/a | |
| mean | 200 | 210 | | 12 | | | |
| SEM | 7 | 21 | | 2 | | | |

ADi-100 1:2 Tx group
Diabetic “Non-responders”

| | | | | | | | | |
|---|---|---|---|---|---|---|---|---|
| 1 | 284 | 191 | 1 | 27 | 209 | 18 | negative | 511 |
| 2 | 214 | 139 | 2 | 27 | 168 | 29 | negative | 351 |

Non-diabetic “Responders”

| | | | | | | | |
|---|---|---|---|---|---|---|---|
| 3 | 211 | 243 | 2 | 20 | n.d. | + | 150 |
| 4 | 185 | 179 | 1 | 7 | n.d. | + | 133 |
| 5 | 286 | 192 | 0 | 6 | n.d. | + | 139 |
| 6 | 212 | 159 | 0 | 13 | n.d. | + | 174 |
| 7 | 202 | 197 | 0 | 3 | n.d. | n/a | 135 |
| 8 | 202 | 180 | 1 | 3 | n.d. | + | 130 |
| 9 | 196 | 188 | 0 | 0 | n.d. | n/a | 151 |
| mean | 213 | 191 | | 7.4 | | | |
| SEM | 13 | 10 | | 2.6 | | | |

Table 1 shows mBG analysis of ADi-100-treated NOD female mice that showed very high hyperglycemia on the first day of treatment (day 0). Female NOD mice were monitored daily for mBG in which each mouse received the first ADi-100 dose (day 0) when mBG was ≥180 mg/dL on at least two occasions or when the first occurrence of mBG was ≥200 mg/dL. Mice received weekly ADi-100 injections thereafter for a total of five injections. Daily mBG monitoring continued and mice were diagnosed with diabetes when ≥300 mg/dL on 2 occasions at least 7 days apart (^{a} values denote age at the first of the 2 mBG measurements). The study ended at day 35, which was when 100% incidence of diabetes occurred in the untreated group (see FIG. 2 for diabetes incidence values). ^{b} p=0.008 (two-tailed unpaired Wilcoxon test) for mean age comparison and p<0.001 (Poisson regression) for mean mBG occurrences comparison to the respective means of non-diabetic responder mice 6-10 in the ADi-100 1:4 group. The untreated control group (n=12) showed a mean±SEM mBG and age on day 0 of 282±29 mg/dL and 120±9 days, respectively, and age at type I diabetes (T1D) diagnosis of 136±12 days. n.d., not diabetic. ^{c} Animals were euthanized at the end of the experiment and pancreata harvested and stained for insulin (see examples of positive and negative insulin staining in FIG. 4).

Collectively, these results demonstrate that ADi-100 induced tol-DC subset migration to draining lymph nodes and that it was strongly efficacious in reversing hyperglycemia and preventing the onset of diabetes in two independent studies; a mechanism that was antigen-specific and relied on the apoptosis-inducing factor, BAX, because neither plasmid alone was efficacious. Importantly, the robust efficacy of ADi-100 is evident in the reproducible results of experiments conducted at two different institutions, a concept that has been raised by the T1D research community [14]. Enhanced efficacy was achieved by increasing the BAX content in the ADi-100 1:2 formulation while proportionally decreasing msGAD55 content to maintain a total dose of 50 μg for comparison with the ADi-100 1:4 formulation. Furthermore, the msGAD55 plasmid was hyper-methylated at CpG motifs to avoid inducing inflammatory signaling, but the BAX plasmid was not hyper-methylated (i.e., was hypo-methylated) to ensure that CMV promoter activity was not compromised [8]. While it may appear counterintuitive that increasing such hypo-methylated plasmid content led to enhanced efficacy, it has been demonstrated that a relatively small amount of unmethylated CpG oligonucleotide added to a tolerogenic immunotherapy can increase expression of the anti-inflammatory cytokine, IL-10, to promote tol-DC and Treg cell development and immune tolerance [15]. Moreover, the hyper-methylation used in developing ADi-100 is analogous to the single-plasmid immunotherapy (expressing proinsulin II) containing recombinantly modified CpG to CpC motifs to avoid inducing inflammation [16], which reversed hyperglycemic NOD mice in addition to showing promising efficacy in T1D clinical trials [17].

Immunotherapies containing different tolerance delivery systems (TDSs) and autoantigens have been shown to prevent diabetes when administered to young pre-hyperglycemic NOD mice, which is similar to Stage 1 in human T1D (i.e., autoantibody positive titers with no signs of dysglycemia; reviewed in [18]). However, there are very few published studies demonstrating that such immunotherapies (as monotherapies) can “reverse hyperglycemia” (i.e., Stage 2) in NOD mice [19]. Several non-specific immunomodulatory agents, such as anti-CD3 mAb, have successfully reversed hyperglycemia in NOD mice, either alone or in combination with an immunotherapy [13,19-21] and have recently been shown to be effective at delaying insulin production loss in pre-diabetic (i.e., dysglycemia, Stage 2) subjects [22]. However, these non-specific therapies may not induce durable tolerance and thus would require long-term dosing with associated safety concerns. DNA-based immunotherapies that contain proinsulin II or secreted GAD, such as our ADi-100 [7,8], have shown success in reversing hyperglycemia in NOD mice when used as monotherapies. A bivalent IgG Fc-MHC/GAD65 fusion protein, DEF-GAD, has also demonstrated such efficacy [23]. The striking effectiveness of these monotherapies to reverse hyperglycemia may be due to prolonged antigen presence in vivo combined with the unique features of each TDS.

Female NOD mice spontaneously developed diabetic hyperglycemia with an incidence of <100%, depending on the colony and laboratory; i.e., usually 70% to 90% incidence [24]. Such unpredictability can be statistically accounted for in “disease prevention” studies with young non-diabetic mice by increasing the number per cohort. However, fewer mice can be used in “hyperglycemia reversal” studies if mice are selected based on the likelihood of developing diabetes. An empirically derived hyperglycemic threshold of 180 mg/dL mBG predictably led to the development of diabetes, which was the upper limit of the true normal mBG range derived from female mice that never developed disease. Indeed, this threshold model was confirmed with the 100% incidence of diabetes in the untreated control group of 12 mice. The accurate prediction of diabetes development in female NOD mice using this threshold is consistent with others who derived a normal mBG range <170 mg/mL or <175 mg/dL and used a diabetes diagnosis of two consecutive values 300 mg/dL or ≥400 mg/dL, respectively (almost all diabetic mice in our study were terminated at mBG ≥500 mg/dL). While these glycemic stages of NOD mice may not translate exactly to those of human T1D, it is clear that ADi-100 could target treatment during clinically detectable dysglycemia (i.e., Stage 2, including hyperglycemia [25]) prior to overt clinical diabetes (Stage 3).

Several prevention or intervention clinical trials with immunotherapies have generally shown disappointing outcomes of preserving insulin production (i.e., stimulated C peptide) and improving glycemic measures (HbA1c and insulin usage) [26]. Most of these immunotherapies consisted of only autoantigens delivered via oral or mucosal (intranasal) routes which could be considered weak TDSs, or of other weak or irrelevant TDSs such as Alum (e.g., GAD-Alum; Diamyd Therapeutics; [27]) or incomplete Freund's adjuvant (IFA) [28,29]. Alum may not be the most effective TDS because it does not appear to induce focused Treg responses, but rather can induce significant Th2 responses and even pathogenic Th1 and Th17 responses (reviewed in [30,31]). Of note, GAD-Alum (Diamyd Therapeutics) has been evaluated in several Phase I and II trials with anti-GAD65 antibody positive (Stage 2) or new-onset (Stage 3) subjects and showed trends toward preservation of residual insulin secretion, especially in subjects with late-onset autoimmune diabetes of adulthood (LADA) [27], but failed this trend in Phase III trials [32,33]. This clinical experience underscores a major problem in the preclinical development of immuotherapies in that GAD-Alum was never tested in animal models prior to clinical evaluation, and positive outcomes of GAD65 efficacy evaluations in NOD mouse efficacy studies were in a “prevention” setting with young (4- to 6-week-old) NOD mice but did not show reversal of the hyperglycemic Stage 2 condition [30]. Interestingly, in a prospective NOD study, the clinical GAD-Alum preparation did not prevent diabetes in the NOD mouse model [30]. Therefore, it is important to develop more regulatory-specific and potent TDSs such as soluble or particulate tolerance vehicles (e.g., nanoparticles, microspheres, and liposomes) containing different tolerogenic agents such as rapamycin, aryl-hydrocarbon receptor ligands, retinoic acid, vitamin D3, and cytokines such as interleukin (10-10 and transforming growth factor (TGF)-β[31,34]. Other TDSs are of a cellular nature in which tol-DCs or Tregs produced ex vivo are reintroduced in vivo [35,36], or are genetically modified gastrointestinal bacterial strains expressing autoantigen and tolerogenic cytokines [37]. Note that apoptotic tolerance vehicles are in this cellular class of TDSs.

Apoptotic-based immunotherapies use a “natural” rather than synthetic tolerance system that avoids the risk of inducing pathogenic autoimmune responses due to the non-inflammatory tolerogenic nature of apoptotic cells (unlike synthetic particles that have a tendency to trigger inflammatory processes [38]). Indeed, there is currently a significant interest in apoptotic-based immunotherapy development using different approaches. One such immunotherapy is a soluble therapeutic comprised of recombinant autoantigen conjugated to a linker molecule that selectively binds erythrocytes (i.e., red blood cells, RBC) via the surface marker, glycophorin A, and upon systemic delivery has shown potent efficacy in preventing diabetes in NOD mice [5,39]. Once autoantigen-bound RBCs enter their natural apoptotic process (eryptosis for non-nucleated RBCs), tolerogenic APCs recognize and process them for interaction with T cells. Note that RBCs have an exceptionally high turnover rate of about 100 billion cells per day, thus potentially delivering high levels of autoantigen-bound apoptotic vesicles to tolerogenic APCs with each dose of the ASI. Another RBC-based apoptotic therapy using the transpeptidase, sortase, to covalently attach autoantigens to RBCs ex vivo prior to reinfusion also showed efficacy in preventing diabetes in NOD mice [6]. In addition, ex vivo chemically-induced apoptosis of mouse splenocytes or human peripheral blood mononuclear cells (PBMCs) [4] demonstrated efficacy in the autoimmune conditions of experimental autoimmune encephalomyelitis and T1D in mice and multiple sclerosis in human trials [40]. Others are using liposomes containing tolerogenic apoptosis mimicry substances such as phosphatidylserine to deliver autoantigen to tol-DCs from human T1D subjects [41].

The disclosed methods are not only highly effective, but have other beneficial qualities such as utilization of a non-cell therapeutic approach, low cost of production, favorable storage profile, and the ability to frequently dose over a long period of time to achieve tolerance.

### REFERENCES

1. Buckner, J. H.; Nepom, G. T. Obstacles and opportunities for targeting the effector T cell response in type 1 diabetes. J. Autoimmun. 2016, 71, 44-50, doi:10.1016/j.jaut.2016.02.009.
2. Hull, C. M.; Peakman, M.; Tree, T. I. M. Regulatory T cell dysfunction in type 1 diabetes: what's broken and how can we fix it? Diabetologia 2017, 60, 1839-1850, doi:10.1007/s00125-017-4377-1.
3. Li, A. F.; Escher, A. DNA vaccines for transplantation. Expert Opin. Biol. Ther 2010, 10, 903-915, doi:10.1517/14712591003796546.
4. Getts, D. R.; McCarthy, D. P.; Miller, S. D. Exploiting apoptosis for therapeutic tolerance induction. J. Immunol. 2013, 191, 5341-5346, doi:10.4049/jimmunol.1302070.
5. Grimm, A. J.; Kontos, S.; Diaceri, G.; Quaglia-Thermes, X.; Hubbell, J. A. Memory of tolerance and induction of regulatory T cells by erythrocyte-targeted antigens. Sci. Rep. 2015, 5, 15907, doi:10.1038/srep15907.
6. Pishesha, N.; Bilate, A. M.; Wibowo, M. C.; Huang, N. J.; Li, Z.; Dhesycka, R.; Bousbaine, D.; Li, H.; Patterson, H. C.; Dougan, S. K., et al. Engineered erythrocytes covalently linked to antigenic peptides can protect against autoimmune disease. Proc. Natl. Acad. Sci. USA 2017, 114, 3157-3162, doi:10.1073/pnas.1701746114.
7. Li, A.; Ojogho, O.; Franco, E.; Baron, P.; Iwaki, Y.; Escher, A. Pro-apoptotic DNA vaccination ameliorates new onset of autoimmune diabetes in NOD mice and induces foxp3+ regulatory T cells in vitro. Vaccine 2006, 24, 5036-5046, doi:10.1016/j.vaccine.2006.03.041.
8. Li, A.; Chen, J.; Hattori, M.; Franco, E.; Zuppan, C.; Ojogho, 0.; Iwaki, Y.; Escher, A. A therapeutic DNA vaccination strategy for autoimmunity and transplantation. Vaccine 2010, 28, 1897-1904, doi:10.1016/j.vaccine.2009.10.090.
9. Li, A. F.; Hough, J.; Henderson, D.; Escher, A. Co-delivery of pro-apoptotic BAX with a DNA vaccine recruits dendritic cells and promotes efficacy of autoimmune diabetes prevention in mice. Vaccine 2004, 22, 1751-1763, doi:10.1016/j.vaccine.2003 0.10.049.
10. Yip, L.; Taylor, C.; Whiting, C. C.; Fathman, C. G. Diminished adenosine A1 receptor expression in pancreatic alpha-cells may contribute to the pathology of type 1 diabetes. Diabetes 2013, 62, 4208-4219, doi:10.2337/db13-0614.
11. Hopp, A. K.; Rupp, A.; Lukacs-Kornek, V. Self-antigen presentation by dendritic cells in autoimmunity. Front. Immunol. 2014, 5, 55, doi:10.3389/fimmu.2014.00055.
12. Takenaka, M. C.; Quintana, F. J. Tolerogenic dendritic cells. Semin Immunopathol 2017, 39, 113-120, doi:10.1007/s00281-016-0587-8.
13. Mathews, C. E.; Xue, S.; Posgai, A.; Lightfoot, Y. L.; Li, X.; Lin, A.; Wasserfall, C.; Haller, M. J.; Schatz, D.; Atkinson, M. A. Acute Versus Progressive Onset of Diabetes in NOD Mice: Potential Implications for Therapeutic Interventions in Type 1 Diabetes. Diabetes 2015, 64, 3885-3890, doi:10.2337/db15-0449.
14. Shoda, L. K.; Young, D. L.; Ramanuj an, S.; Whiting, C. C.; Atkinson, M. A.; Bluestone, J. A.; Eisenbarth, G. S.; Mathis, D.; Rossini, A. A.; Campbell, S. E., et al. A comprehensive review of interventions in the NOD mouse and implications for translation. Immunity 2005, 23, 115-126, doi:10.1016/j.immuni.2005.08.002.
15. Yoon, Y. M.; Lewis, J. S.; Carstens, M. R.; Campbell-Thompson, M.; Wasserfall, C. H.; Atkinson, M. A.; Keselowsky, B. G. A combination hydrogel microparticle-based vaccine prevents type 1 diabetes in non-obese diabetic mice. Sci. Rep. 2015, 5, 13155, doi:10.1038/srep13155.
16. Solvason, N.; Lou, Y. P.; Peters, W.; Evans, E.; Martinez, J.; Ramirez, U.; Ocampo, A.; Yun, R.; Ahmad, S.; Liu, E., et al. Improved efficacy of a tolerizing DNA vaccine for reversal of hyperglycemia through enhancement of gene expression and localization to intracellular sites. J. Immunol. 2008, 181, 8298-8307.
17. Roep, B. O.; Solvason, N.; Gottlieb, P. A.; Abreu, J. R. F.; Harrison, L. C.; Eisenbarth, G. S.; Yu, L.; Leviten, M.; Hagopian, W. A.; Buse, J. B., et al. Plasmid-encoded proinsulin preserves C-peptide while specifically reducing proinsulin-specific CD8(+) T cells in type 1 diabetes. Sci. Transl. Med. 2013, 5, 191ra182, doi:10.1126/scitranslmed.3006103.
18. Krishnamurthy, B.; Selck, C.; Chee, J.; Jhala, G.; Kay, T. W. Analysis of antigen specific T cells in diabetes—Lessons from pre-clinical studies and early clinical trials. J. Autoimmun. 2016, 71, 35-43, doi:10.1016/j jaut.2016.03.018.
19. Ben Nasr, M.; D′Addio, F.; Usuelli, V.; Tezza, S.; Abdi, R.; Fiorina, P. The rise, fall, and resurgence of immunotherapy in type 1 diabetes. Pharm. Res. 2015, 98, 31-38, doi: 10.1016/j.phrs.2014.07.004.
20. Takiishi, T.; Cook, D. P.; Korf, H.; Sebastiani, G.; Mancarella, F.; Cunha, J. P.; Wasserfall, C.; Casares, N.; Lasarte, J. J.; Steidler, L., et al. Reversal of Diabetes in NOD Mice by Clinical-Grade Proinsulin and IL-10-Secreting Lactococcus lactis in Combination With Low-Dose Anti-CD3 Depends on the Induction of Foxp3-Positive T Cells. Diabetes 2017, 66, 448-459, doi:10.2337/db15-1625.
21. Gill, R. G.; Pagni, P. P.; Kupfer, T.; Wasserfall, C. H.; Deng, S.; Posgai, A.; Manenkova, Y.; Bel Hani, A.; Straub, L.; Bernstein, P., et al. A Preclinical Consortium Approach for Assessing the Efficacy of Combined Anti-CD3 Plus IL-1 Blockade in Reversing New-Onset Autoimmune Diabetes in NOD Mice. Diabetes 2016, 65, 1310-1316, doi:10.2337/db15-0492.
22. Herold, K. C.; Bundy, B. N.; Long, S. A.; Bluestone, J. A.; DiMeglio, L. A.; Dufort, M. J.; Gitelman, S. E.; Gottlieb, P. A.; Krischer, J. P.; Linsley, P. S., et al. An Anti-CD3 Antibody, Teplizumab, in Relatives at Risk for Type 1 Diabetes. N Engl. J. Med. 2019, 381, 603-613, doi:10.1056/NEJMoa1902226.
23. Lin, M.; Stoica-Nazarov, C.; Surls, J.; Kehl, M.; Bona, C.; Olsen, C.; Brumeanu, T. D.; Casares, S. Reversal of type 1 diabetes by a new MEW II-peptide chimera: “Single-epitope-mediated suppression” to stabilize a polyclonal autoimmune T-cell process. Eur. J. Immunol. 2010, 40, 2277-2288, doi:10.1002/eji.200940094.
24. van Belle, T. L.; Coppieters, K. T.; von Herrath, M. G. Type 1 diabetes: Etiology, immunology, and therapeutic strategies. Physiol. Rev. 2011, 91, 79-118, doi:10.1152/physrev.00003.2010.
25. Insel, R. A.; Dunne, J. L.; Atkinson, M. A.; Chiang, J. L.; Dabelea, D.; Gottlieb, P. A.; Greenbaum, C. J.; Herold, K. C.; Krischer, J. P.; Lernmark, A., et al. Staging presymptomatic type 1 diabetes: A scientific statement of JDRF, the Endocrine Society, and the American Diabetes Association. Diabetes Care 2015, 38, 1964-1974, doi:10.2337/dc15-1419.
26. Skyler, J. S. Prevention and reversal of type 1 diabetes-past challenges and future opportunities. Diabetes Care 2015, 38, 997-1007, doi:10.2337/dc15-0349.
27. Agardh, C. D.; Cilio, C. M.; Lethagen, A.; Lynch, K.; Leslie, R. D.; Palmer, M.; Harris, R. A.; Robertson, J. A.; Lernmark, A. Clinical evidence for the safety of GAD65 immunomodulation in adult-onset autoimmune diabetes. J. Diabetes Complications 2005, 19, 238-246, doi:10.1016/j.jdiacomp.2004.12.003.
28. Ryden, A. K.; Wesley, J. D.; Coppieters, K. T.; Von Herrath, M. G. Non-antigenic and antigenic interventions in type 1 diabetes. Hum. Vaccin Immunother 2014, 10, 838-846.
29. Clemente-Casares, X.; Tsai, S.; Huang, C.; Santamaria, P. Antigen-specific therapeutic approaches in Type 1 diabetes. Cold Spring Harb Perspect Med. 2012, 2, a007773, doi:10.1101/cshperspect.a007773.
30. Von Herrath, M.; Peakman, M.; Roep, B. Progress in immune-based therapies for type 1 diabetes. Clin. Exp. Immunol. 2013, 172, 186-202, doi:10.1111/cei.12085.
31. Keijzer, C.; van der Zee, R.; van Eden, W.; Broere, F. Treg inducing adjuvants for therapeutic vaccination against chronic inflammatory diseases. Front. Immunol 2013, 4, 245, doi:10.3389/fimmu.2013.00245.
32. Ludvigsson, J.; Krisky, D.; Casas, R.; Battelino, T.; Castano, L.; Greening, J.; Kordonouri, O.; Otonkoski, T.; Pozzilli, P.; Robert, J. J., et al. GAD65 antigen therapy in recently diagnosed type 1 diabetes mellitus. N Engl. J. Med. 2012, 366, 433-442, doi:10.1056/NETMoa1107096.
33. Wherrett, D. K.; Bundy, B.; Becker, D. J.; DiMeglio, L. A.; Gitelman, S. E.; Goland, R.; Gottlieb, P. A.; Greenbaum, C. J.; Herold, K. C.; Marks, J. B., et al. Antigen-based therapy with glutamic acid decarboxylase (GAD) vaccine in patients with recent-onset type 1 diabetes: A randomised double-blind trial. Lancet 2011, 378, 319-327, doi:10.1016/S0140-6736(11)60895-7.
34. Serra, P.; Santamaria, P. Nanoparticle-based autoimmune disease therapy. Clin. Immunol. 2015, 160, 3-13, doi:10.1016/j.clim.2015.02.003.
35. Giannoukakis, N.; Trucco, M. Dendritic cell therapy for Type 1 diabetes suppression. Immunotherapy 2012, 4, 1063-1074, doi:10.2217/imt.12.76.
36. Seay, H. R.; Putnam, A. L.; Cserny, J.; Posgai, A. L.; Rosenau, E. H.; Wingard, J. R.; Girard, K. F.; Kraus, M.; Lares, A. P.; Brown, H. L., et al. Expansion of Human Tregs from Cryopreserved Umbilical Cord Blood for GMP-Compliant Autologous Adoptive Cell Transfer Therapy. Mol. Ther Methods Clin. Dev. 2017, 4, 178-191, doi:10.1016/j.omtm.2016.12.003.
37. Robert, S.; Gysemans, C.; Takiishi, T.; Korf, H.; Spagnuolo, I.; Sebastiani, G.; Van Huynegem, K.; Steidler, L.; Caluwaerts, S.; Demetter, P., et al. Oral delivery of glutamic acid decarboxylase (GAD)-65 and IL10 by Lactococcus lactis reverses diabetes in recent-onset NOD mice. Diabetes 2014, 63, 2876-2887, doi:10.2337/db13-1236.
38. Hartwell, B. L.; Antunez, L.; Sullivan, B. P.; Thati, S.; Sestak, J. O.; Berkland, C. Multivalent nanomaterials: Learning from vaccines and progressing to antigen-specific immunotherapies. J. Pharm. Sci. 2015, 104, 346-361, doi:10.1002/jps.24273.
39. Kontos, S.; Kourtis, I. C.; Dane, K. Y.; Hubbell, J. A. Engineering antigens for in situ erythrocyte binding induces T-cell deletion. Proc. Natl. Acad. Sci. USA 2013, 110, E60-68, doi:10.1073/pnas.1216353110.
40. Lutterotti, A.; Yousef, S.; Sputtek, A.; Sturner, K. H.; Stellmann, J. P.; Breiden, P.; Reinhardt, S.; Schulze, C.; Bester, M.; Heesen, C., et al. Antigen-specific tolerance by autologous myelin peptide-coupled cells: A phase 1 trial in multiple sclerosis. Sci. Transl. Med. 2013, 5, 188ra175, doi:10.1126/scitranslmed.3006168.
41. Rodriguez-Fernandez, S.; Pujol-Autonell, I.; Brianso, F.; Perna-Barrull, D.; Cano-Sarabia, M.; Garcia-Jimeno, S.; Villalba, A.; Sanchez, A.; Aguilera, E.; Vazquez, F., et al. Phosphatidylserine-Liposomes Promote Tolerogenic Features on Dendritic Cells in Human Type 1 Diabetes by Apoptotic Mimicry. Front. Immunol. 2018, 9, 253, doi:10.3389/fimmu.2018.00253.

1-62. (canceled)

63. A method of suppressing type 1 diabetes onset in a patient with hyperglycemia, the method comprising administering a vector system comprising:
(a) a first vector comprising:
a first expression cassette comprising a polynucleotide encoding BCL2 associated X apoptosis regulator (BAX); and
a promoter operably linked to the polynucleotide encoding BAX; and
(b) a second vector comprising a second expression cassette hypermethylated at CpG motifs and comprising:
a polynucleotide encoding a secreted form of glutamic acid decarboxylase 65 (GAD65) encoded by mdGAD; and
a promoter operably linked to the polynucleotide encoding GAD65,
in an amount therapeutically effective in delaying type 1 diabetes onset in at least 80% of patients treated by at least one month as compared to untreated patients with similar hyperglycemias,
wherein the first vector and second vector are administered in a ratio in a range or 1:1 and 1:8.

64. The method of claim 63, wherein the first expression cassette comprises a CMV promoter or an SV-40 promoter operably linked to the polynucleotide encoding the BAX.

65. The method of claim 63, wherein the patient has mild hyperglycemia.

66. The method of claim 63, wherein the patient has moderate hyperglycemia.

67. The method of claim 63, wherein the patient has severe hyperglycemia.

68. The method of claim 63, wherein a patient has type 1 diabetes if the patient has an amount of insulin-producing pancreatic beta cells less than 50% of a reference amount of pancreatic beta cells for a patient without type 1 diabetes or hyperglycemia.

69. The method of claim 63, wherein the vector system is administered intradermally or mucosally.

70. The method of claim 63, wherein the method further comprises increasing numbers of tolerogenic dendritic cells and/or GAD-specific regulatory T cells in the patient in response to administering the vector system.

71. A method of reversing hyperglycemia in a patient with hyperglycemia, the method comprising administering a vector system comprising:
(a) a first vector comprising:
a first expression cassette comprising a polynucleotide encoding BCL2 associated X apoptosis regulator (BAX); and
a promoter operably linked to the polynucleotide encoding BAX; and
(b) a second vector comprising a second expression cassette hypermethylated at CpG motifs and comprising:
a polynucleotide encoding a secreted form of glutamic acid decarboxylase 65 (GAD65) encoded by mdGAD; and
a promoter operably linked to the polynucleotide encoding GAD65,
in an amount therapeutically effective to cause hyperglycemia to not be detectable in at least 80% of patients treated for at least one month subsequent to treatment,
wherein the first vector and second vector are administered in a ratio in a range or 1:1 and 1:8.

72. The method of claim 71, wherein the first expression cassette comprises a CMV promoter or an SV-40 promoter operably linked to the polynucleotide encoding the BAX.

73. The method of claim 71, wherein the patient has mild hyperglycemia.

74. The method of claim 71, wherein the patient has moderate hyperglycemia.

75. The method of claim 71, wherein the patient has severe hyperglycemia.

76. The method of claim 71, wherein the vector system is administered intradermally or mucosally.

77. The method of claim 71, wherein the method further comprises increasing numbers of tolerogenic dendritic cells and/or GAD-specific regulatory T cells in the patient in response to administering the vector system.

78. A method of increasing numbers of tolerogenic dendritic cells and GAD-specific regulatory T cells in a patient having hyperglycemia, the method comprising administering a vector system comprising:
(a) a first vector comprising a first expression cassette comprising:
a polynucleotide encoding BCL2 associated X apoptosis regulator (BAX); and
a promoter operably linked to the polynucleotide encoding BAX; and
(b) a second vector comprising a second expression cassette hypermethylated at CpG motifs and comprising:
a polynucleotide encoding a secreted form of glutamic acid decarboxylase 65 (GAD65) encoded by mdGAD; and
a promoter operably linked to the polynucleotide encoding GAD65,
in an amount therapeutically effective to increase numbers of tolerogenic dendritic cells and GAD-specific regulatory T cells in at least 80% of patients treated for at least one month subsequent to treatment,
wherein the first vector and second vector are administered in a ratio in a range or 1:1 and 1:8.

79. The method of claim 78, wherein the proportion of CD8α⁺ tolerogenic dendritic cells to the total CD11c⁺ dendritic cell population in draining lymph nodes is increased about 13-fold.

80. The method of claim 78, wherein the proportion of CD11b⁺/CD103⁺ tolerogenic dendritic cells to the total CD11c⁺ dendritic cell population in draining lymph nodes is increased about 2-fold.

81. The method of claim 78, wherein the proportion of CD207⁺ tolerogenic dendritic cells to the total CD11c⁺ dendritic cell population in draining lymph nodes is increased about 2.5-fold.

82. The method of claim 78, wherein the vector system is administered intradermally or mucosally.
